SEQUENCE LISTING (26 sequences)

SEQ ID NO: 1 - protein, 12 aa, Unknown
  CHAIN: murine lactate dehydrogenase-C polypeptide
EQLIQNLVPEDK

SEQ ID NO: 2 - protein, 15 aa, Unknown
  CHAIN: murine lactate dehydrogenase-C polypeptide
GLADELALVDADTDK

SEQ ID NO: 3 - protein, 18 aa, Unknown
  CHAIN: murine lactate dehydrogenase-C polypeptide
GEALDLDQHGSLFLSTPK

SEQ ID NO: 4 - protein, 35 aa, Unknown
  CHAIN: murine lactate dehydrogenase-C polypeptide
LGVNPTSCHGWVLGEHGDSSVPIWSGVNVAGVTLK

SEQ ID NO: 5 - protein, 10 aa, Unknown
  CHAIN: murine lactate dehydrogenase-C polypeptide
SLNPAIGTDK

SEQ ID NO: 6 - protein, 12 aa, Unknown
  CHAIN: murine lactate dehydrogenase-C polypeptide
QVVEGGYEVLDK

SEQ ID NO: 7 - protein, 21 aa, Unknown
  CHAIN: murine lactate dehydrogenase-C polypeptide
EEVFLSIPCVLGESGITDFVK

SEQ ID NO: 8 - protein, 13 aa, Unknown
  CHAIN: murine lactate dehydrogenase-C polypeptide
VNMTAEEEGLLLK

SEQ ID NO: 9 - protein, 14 aa, Unknown
  CHAIN: murine lactate dehydrogenase-C polypeptide
RMVSGQTRLDLLQR

SEQ ID NO: 10 - protein, 18 aa, Unknown
  CHAIN: egg zona pellucida glycoprotein
PVTQSGPLRLELRLATDK

SEQ ID NO: 11 - protein, 7 aa, Unknown
  CHAIN: egg zona pellucida glycoprotein
FGIHGPR

SEQ ID NO: 12 - protein, 14 aa, Unknown
  CHAIN: immunocontraceptive polypeptide
LNSSSSQFQIHGPR

SEQ ID NO: 13 - protein, 70 aa, Unknown
  CHAIN: immunocontraceptive polypeptide
CPKPDHTVTPDFYLAPPTTPEPFTPHAFALHPIPDHTLAGSGHTGLTTLYPEQSFIHPTPAPPSLGPGPA

SEQ ID NO: 14 - protein, 21 aa, Unknown
  CHAIN: immunocontraceptive polypeptide
WFLQSDNEDARIHSLYGMISC

SEQ ID NO: 15 - protein, 15 aa, Unknown
  CHAIN: immunocontraceptive polypeptide
ALNNRFQIKGVELKS

SEQ ID NO: 16 - protein, 11 aa, Unknown
  CHAIN: immunocontraceptive polypeptide
NCAYKTTQANK

SEQ ID NO: 17 - protein, 43 aa, Unknown
  CHAIN: immunocontraceptive polypeptide
CQADSGLQNRLALFTFPNISETNVTYLFGHEENSTEHAMKGVC

SEQ ID NO: 18 - protein, 13 aa, Unknown
  CHAIN: murine lactate dehydrogenase-C polypeptide
QLINLVPEDKLSR

SEQ ID NO: 19 - protein, 13 aa, Unknown
  CHAIN: rat lactate dehydrogenase-C polypeptide
QLINLAPDEKQSR

SEQ ID NO: 20 - protein, 13 aa, Unknown
  CHAIN: human lactate dehydrogenase-C polypeptide
QLIKLIEDDENSQ

SEQ ID NO: 21 - protein, 13 aa, Unknown
  CHAIN: hamster lactate dehydrogenase-C polypeptide
QLINLTQEDKTSR

SEQ ID NO: 22 - protein, 13 aa, Unknown
  CHAIN: pig lactate dehydrogenase-C polypeptide
QLINLIEDEVSQR

SEQ ID NO: 23 - protein, 13 aa, Unknown
  CHAIN: fox lactate dehydrogenase-C polypeptide
QLENLIEEDKISQ

SEQ ID NO: 24 - protein, 13 aa, Unknown
  CHAIN: Pigeon lactate dehydrogenase-C polypeptide
QLIMTPITAQIKV

SEQ ID NO: 25 - protein, 13 aa, Unknown
  CHAIN: Killifish lactate dehydrogenase-C polypeptide
HKLITPLACSSPE

SEQ ID NO: 26 - protein, 13 aa, Unknown
  CHAIN: frog lactate dehydrogenase-C polypeptide
NLITNVCQDKAAA

(19) United States
(12) Patent Application Publication
(10) Pub. No.: US 2005/0191318 A1
(43) Pub. Date: Sep. 1, 2005

(54) DNA immunocontraceptive vaccines and uses thereof

(76) Inventors: G. Barrie Kitto, Austin, TX (US); Daniel C. Hirschhorn, New York, NY (US)

Correspondence Address: FULBRIGHT & JAWORSKI, L.L.P., 600 CONGRESS AVENUE, SUITE 2400, AUSTIN, TX, 78701, US

(21) Appl. No.: 10/789,729

(22) Filed: Feb. 27, 2004

(51) Int. Cl. A61K 39/02; C12N 1/21

(52) U.S. Cl. 424/200.1; 435/252.3

(57) ABSTRACT

The present invention provides an immunocontraceptive vaccine comprising a recombinant bacterial host that has been modified to produce egg- or sperm-specific polypeptide such as the sperm-specific protein lactate dehydrogenase-C (LDH-C). When animals such as rodents eat these bacteria, their immune systems produce antibodies that attack their sperms. Not only would the males have less viable sperm, the females would also have antibodies to sperm entering their reproductive systems. Immunocontraception is an attractive method for reducing the population size of animals with high fecundity, and sterilizing animals using such immunocontraceptives can reduce targeted animal populations to acceptable levels in an efficient, cost-effective, humane and, importantly, a species-specific manner.

### BACKGROUND OF THE INVENTION

1. Field of the Invention

The present invention relates generally to the field of immunocontraceptives. More specifically, the present invention relates to methods of controlling animal populations using immunocontraceptives expressed as DNA vaccines.

2. Description of the Related Art

Rodent pests have infiltrated human activities and caused great economic and social impact. They can cause massive destruction of crops and foodstuffs and spread fatal diseases to people and domestic animals. The so-called comensal rodents include the Norway rat (Rattus norvegicus), the roof or house rat (Rattus rattus), and the several sub-species of mice Mus musculus (domesticus, spretus, mecedonicus, hortulanus, molossinus, and castaneous).

According to estimates by the Food and Agricultural Organization of the United Nations (FAO), rodents destroy 40 million tons of food annually. This amount is sufficient to feed 130 million people. It has been estimated that some ⅓ to ⅕ of the world's crops are being destroyed by commensal rodents.

In industrialized countries with an overproduction of crops, the damage is inflicted primarily in storage facilities where losses are primarily economic. In contrast, in developing nations where starvation is prominent, crops are being destroyed in the field as well as in storage. An adult rat can consume 20-30 g of grain per day; this means that just 100 rats on a farm will consume at least a ton of food intended for humans or animal fed.

The damage to crop is usually overshadowed by the very serious contamination of grain with feces, urine, and hair. A rat produces on average 40 droppings a day. At this rate, ten rats will produce 146,000 droppings a year. The same amount of rats can produce 54 liters of urine. If only a few droppings or a small amount of urine finds its way to food intended for human consumption it would most likely be rejected. This is because, among other things, waste products are carriers of disease. In addition, rodent carcasses killed by poisons can render useless entire reservoirs of grain and other crops.

Rats, mice, and other rodents are capable of carrying and transmitting a variety of diseases to human and other animals. Viruses rodents transmit cause serious diseases in man with fatality rates up to 50% or more. Some examples include Venezuelan equine encephalitis, tick-born encephalitis, rodent-borne hemorrhagic fevers (Argentine, Bolivian, and Venezuelan), Lassa virus, and Hemorrhagic fever with renal syndrome.

Rodents can transmit a variety of rickettsial diseases using vectors such as ticks, mites, fleas, or mosquitoes. The principal rickettsial diseases that inflict human populations include louse-borne typhus (Rickettsia prowazeki), Rocky Mountain spotted fever, Boutonneuse fever, tustsugamushi diseases (Scrub typhus), and murine typhus (Rickettsia typhi). Rodents are also reservoirs of a great number of bacterial diseases. In fact, they are particularly known for carrying bacteria such as Salmonella, plague, Lyme disease, Borrelia, and leptospirosis.

Mechanical/physical rodent control includes shooting, flooding burrows with water, use of ultrasound or electromagnetic waves. Using high tech devices such as ultrasound or electromagnetic waves is not only an extremely expensive alternative but also has never been shown to produce consistent results. Trapping will produce some results under very limited conditions such as a house. However, trapping does not protect crops and it is only used in conjunction with chemical rodenticides to remove surviving animals from treated areas.

Biological rodent control refers to the use of predators, pathogens, and parasites that induces mortality or migration of rodents. Except for domestic cats, all other natural predators have been shown to be useless in rodent control. This is because the population size of the predator is directly proportional to the population size of the target species. Since the rodent's reproduction rate is much higher than its predator's rate, by the time the predator numbers are large enough to control an infestation enormous damage has already been done.

Many unsuccessful attempts to introduce pathogens for pest control have been attempted. In fact only one has been proven successful but not in rodents. The myxoma virus was introduced into Australia to control rabbits some 50 years ago. Similar approaches using Salmonella enteritis to control rodents in many countries have been attempted since the turn of the century in the form of baits called Ratin. Despite its initial success, concerns about safety have led many to abandon this approach.

Ecological rodent control refers to changing the environment of a particular infested area such that it is not attractive to rodents anymore. This means that the environment has to be sufficiently different to make rodents uncomfortable. Ecological control has only been shown to be effective in combination with other methods of control.

Genetical rodent control refers primarily to the possibility of altering the normal gene pool within a population by introducing a deleterious gene or introducing sterile males. For example, introducing genes such as the one causative of the Gruneber Lethal Syndrome that results in the death of 25% of the offspring before puberty has been explored. However, natural selection will operate to eliminate such genes from the population.

Introducing sterilized individuals into the population was tested on several occasions by releasing infertile Norway rat males into the population. It was claimed that some success was achieved since the sterile males produced pseudopregnant females, out competing fertile males. The success of this approach depends on the number of sterile males released, their survival, and the mating system of the target species. Unfortunately, research has been very limited in genetic research since it has proven in the past to be technically difficult, expensive, and has a long time frame. But its sound biological basis, potential species-specificity, and no environmental contamination may one day let it play an important role in rodent control.

Chemical rodent control include repellents or attractants, rodenticides, and chemosterilants. Repellents are not species-specific and can potentially render food or goods with repugnant odor or taste. Attractants have long been used in combination with either traps, poisons, and/or, chemosterilants.

Chemical poisons or rodenticides are the most widely used and efficient of all available methods for rodent control. The introduction of these agents provided society with an extremely powerful tool in the battle against rodents. There is a tremendous selection pressure favoring genetic resistance to a poison that can be rapidly fixed in the population. Eventually, certain poisons were no longer effective to control infestation at all and new chemicals have to be developed. Thus, the use of chemical poisons is not an effective strategy in the long-term because rodents breed rapidly and those that were killed are readily replaced.

It has been suggested that reducing fertility is a more preferable and effective method to control rodents and other mammalian pests. In rodent and other vertebrate pest control, sterilization is considered to have more potential and more impact on a population than conventional killing approaches. For decades, several models and some experimental data have shown that reducing sterility is actually a more effective approach for long-term control.

There are numerous publications on rodent chemosterilants in the 1960's and early 1970's but not much has been done thereafter. The reason for this might be due to several factors. To begin with, there is not a single chemosterilant that can act in both sexes. Moreover, the sterilant has to be supplied continuously and the sterilized rodents will continue to do damage until the end of their days. But perhaps the most important reason that research stopped focusing on chemosterilants is because they share most of the problems that chemical poisons hold, such as high toxicity, lack of species specificity, and the ability to pass through food chains.

The purpose of an anti-fertility vaccine or immunocontraceptive is to induce immune response against proteins from the reproductive system, thereby leading to infertility. Perhaps the widest application of contraceptive vaccines is for the control of rodents and other mammalian species. A successful contraceptive vaccine will have to block fertilization or embryonic development, be species specific, and provoke a sustained immune response. Additionally, an effective mechanism which is cost effective to manufacture and administer for transmitting the vaccine safely throughout the target population must be found.

A fundamental issue in the development of immunocontraceptive agents for the control of wild species is the method of delivery. The prior art is deficient in efficient methods of delivering immunocontraceptives useful in controlling animal populations such as rodents. The present invention fulfills this long-standing need and desire in the art by providing immunocontraceptives expressed as DNA vaccines.

### SUMMARY OF THE INVENTION

The present invention provides a fertility control agent that is cost-effective, humane and species specific for the control of animal populations. The fertility control agent comprises a genetically engineered bacterial host that has been modified to produce the sperm-specific protein lactate dehydrogenase-C (LDH-C). When animals such as rodents eat these modified bacteria, their immune system produces antibodies that attack their sperms. Because the antibodies react to sperm, the instant fertility control agent affects the fertility of both males and females. Not only would the males have less viable sperms, the females would also have antibodies to the sperms entering their reproductive systems. The induced sterility is only maintained as long as the animals ingest the bacteria. Therefore, there is no concern over extinction of the targeted animals or of their predators.

In one embodiment of the present invention, there is provided a genetically modified bacterial host that expresses an immunocontraceptive comprising an egg- or sperm-specific polypeptide. The contraceptive agent is useful in controlling the size of an animal population by inducing sterility in animals that have ingested the genetically-engineered bacteria.

In another embodiment of the present invention, there are provided methods of using these contraceptive agents to control the population size of animals that has reached pest levels.

Other and further aspects, features, and advantages of the present invention will be apparent from the following description of the presently preferred embodiments of the invention. These embodiments are given for the purpose of disclosure.

### BRIEF DESCRIPTION OF THE DRAWINGS

FIG. 1 shows a modeling of rodent population size after treatment with 60% or 80% effective contraceptive. The constraints used were: initial population=20 mice (10 female, 10 male); average lifespan=1 year; gestation time=20 days; fecundity, 5 litters/year, 8 offspring/litter; time to sexual maturity from birth=45 days.

FIG. 2 is a map of pcDNA3.1-LDH-C designed to express the complete sequence of lactate dehydrogenase-C.

FIG. 3 shows PCR results of DNA extracted from a vaccinated mouse. Lane 1, stomach DNA 2 μl; lane 2, stomach 1 μl; lane 3, spleen 2 μl; lane 4, spleen 1 μl; lane 5, pcDNA3-LDH-C 200 ng; lane 6, water (negative control); lane 7; water. Approximately equal amounts of DNA from tissues were subjected to PCR for lactate dehydrogenase-C.

FIG. 4 shows average and individual serum IgG response to DNA-Salmonella vaccination. The IgG was measured by indirect ELISA on days 0, 16, 23, 34, 45, and 87. Each arrow represents the day that the vaccine was administered. The serum was diluted 1:500.

FIG. 5 shows average and individual serum IgA response to DNA-Salmonella vaccination. The IgA was measured by indirect ELISA on days 0, 16, 23, 34, 45, and 87. Each arrow represents the day that the vaccine was administered. The serum was diluted 1:10. Data for the last bleed was not shown.

FIG. 6 shows average vaginal IgA response to DNA-Salmonella vaccination. The IgA was measured by indirect ELISA on days 0, 16, 23, 34, 45, and 87. Each arrow represents the day that the vaccine was administered. The vaginal wash was diluted 1:10.

FIG. 7 shows pooled serum IgG response to DNA-Salmonella vaccination. The ELISAs were performed on pooled serum samples. The arrows represent the day of vaccine administration. The serum was diluted 1:500 and was incubated overnight at 4° C.

FIG. 8 shows pooled serum IgA response to DNA-Salmonella vaccination. The ELISAs were performed on pooled serum samples. The arrows represent the day of vaccine administration. The serum was diluted 1:10 and was incubated overnight at 4° C.

FIG. 9 shows pooled vaginal IgA response to DNA-Salmonella vaccination. The ELISAs were performed on pooled vaginal wash samples (diluted 1:10). The arrows represent the day of vaccine administration.

FIG. 10 shows Th-cell proliferation assays for the Salmonella-DNA vaccine. Titrating amounts of either recombinant lactate dehydrogenase-C or heat-killed Salmonella were used in each group. The legend includes: Black bar, target cells and responder cells from the pcDNA3-LDH-C-treated animals; Red bar, target cells and responder cells from the pcDNA3-GFP treated mice (control); Green bar, only responder cells from the pcDNA3-LDH-C-treated mice (control); Yellow bar, responder cells from the pcDNA3-GFP treated mice (control); Blue bar, mitomycin C-treated antigen presenting cells only (control/baseline). The ELISA is measuring BrdU incorporation.

### DETAILED DESCRIPTION OF THE INVENTION

As used herein, “immunocontraceptive” refers to an immunogenic composition comprising an antigen that can induce immune responses against the cells of an animal's reproductive system, thereby leading to loss of fertility in the treated animal.

The present invention discloses development of new methods for the control of animal populations that reach pest proportion. The present invention incorporates recently developed immunocontraception technologies. The basic premise of immunocontraceptives is to orally immunize animals by using bait formulations containing proteins from the animals' own reproductive system so that immune responses that block fertilization are induced in the animals after ingestion of such baits. Immunocontraception is an attractive method for reducing the population size of animals with high fecundity, and it is believed that sterilizing animals using such immunocontraceptives can reduce targeted animal populations to acceptable levels in an efficient, cost-effective, humane and, importantly, a species-specific manner.

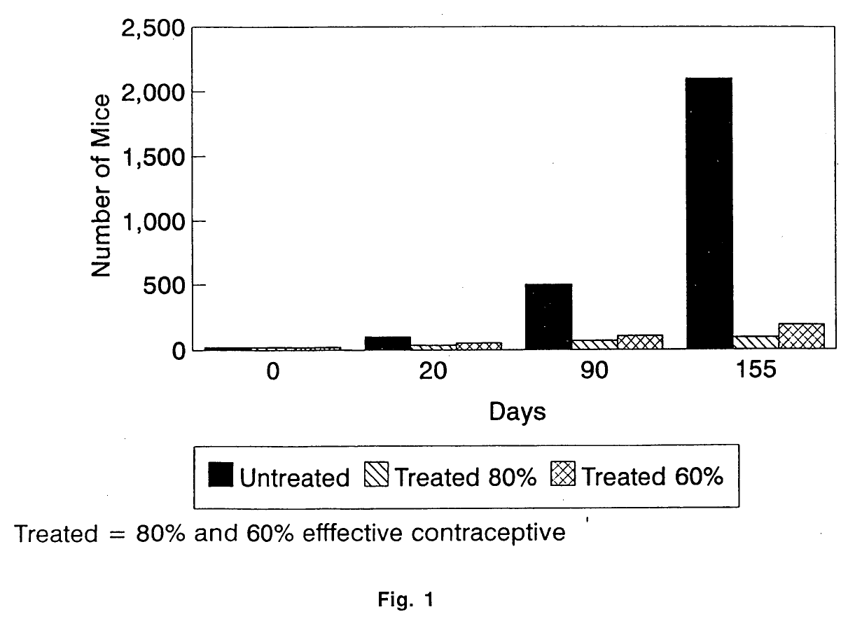

Using some simplifying assumptions in a modeling of contraceptive effects on mouse populations, some calculations were made of how vaccines of 60% and 80% effectiveness might affect the population growth of a small starting population of mice in a habitat without resource constraints. This is a circumstance which might exist when mice initially infest a granary. The results of such modeling are depicted schematically in FIG. 1, dramatically illustrating how an oral contraceptive of even 60% effectiveness can alter the rate of mouse population growth. The constraints used were: initial population=20 mice (10 female, 10 male); average lifespan=1 year; gestation time=20 days; fecundity, 5 litters/year, 8 offspring/litter; time to sexual maturity from birth=45 days.

Lactate Dehydrogenase C4 as an Antigen for Immunocontraception

The best-characterized sperm antigen that has been used to develop a contraceptive vaccine is lactate dehydrogenase-C (LDH-C). All vertebrate tissue contains lactate dehydrogenases (EC 1.1.1.27). This tetrameric protein has multiple isozymes assembled primarily by two subunits, A and B, that can form 5 different molecular forms. Lactate dehydrogenase-C4 (LDH-C4) is a sixth isozyme of lactate dehydrogenase that exclusively expresses in the testis of mammals and other animal species. It represents the most predominant form in the testis and is readily detectable in mature testes displaying active spermatogenesis. The enzyme does not express in other tissue in males and it is completely absent in females. Immunofluorescent studies reveal a remarkable spatio-temporal regulation of the ldh-c gene where it is present in spermatocytes, spermatids, and spermatozoa but not in other non-germinal elements. LDH-C4 differs from the other lactate dehydrogenase isozymes in amino acid composition and in its catalytic properties. Most interestingly, sera raised against LDH-C do not cross-react with the A or B subunits. Rabbit antiserum to mouse LDH-C4 cross reacts with LDH-C4 from other species but never with any isozyme composed of LDH-A or B subunits.

The first reports that lactate dehydrogenase-C can be used as a contraceptive agent come from studies where antibodies were passively transferred to animals. In this experiment, pregnancy suppression occurred in mice treated with rabbit anti-lactate dehydrogenase-C serum. Active immunization of purified lactate dehydrogenase-C produced infertility in female mice, rabbits, and baboons (Lerum and Goldberg, 1974; Goldberg, 1973; Goldberg et al., 1981). Immunized female mice produced 60 pups as opposed to 96 for the control mice after 50 matings (Lerum and Goldberg, 1974). In experiments with rabbits the fertility of the animals was reduced by 60% when mated 40 times (Goldberg, 1973). Lastly, immunized female baboons produced 27 offspring as opposed to 80 by control animals after 30 matings (Goldberg et al., 1981). The contraceptive effects in these model animals correlated with the presence of a specific immune response. Once the immune response faded away, the fertility of the animals reverted to normal fertility levels, proving that the contraceptive effect was reversible. Moreover, this form of contraception showed no impairment of embryonic development or damage to the female reproductive tract.

The availability of complete amino acid sequence information and high-resolution crystal structure for the lactate dehydrogenase-C4 antigen has allowed for B-cell epitope mapping of the molecule and subsequent synthesis of species-specific immunodominant epitope peptides (Kaumaya et al., 1992; Hogrefe et al., 1987). Immunodominant epitopes of lactate dehydrogenase-C4 can be identified by computer algorithms for B-cell epitope prediction (Van Regenmortel and Daney de Marcillac, 1998). The predicted epitopes can be corroborated by sequence comparison with related mammalian lactate dehydrogenase-C immunodominant epitopes. The human, baboon, mouse, and rabbit share the most immunodominant epitope 5-15 amino acids at the N-terminus of the molecule (O'Hern et al., 1995, 1997). Other examples of lactate dehydrogenase-C4 antigenic domains include amino acids 5-17, 44-58, 61-77, 97-110, 101-115, 180-210, 211-220, 231-243, 283-306, and 307-316.

A peptide vaccine consisted of a lactate dehydrogenase-C4 epitope chemically linked to a diphtheria toxoid has been tested in rabbits. The diphtheria toxoid was used with the double purpose of carrier protein and adjuvant. The peptide was further adapted for baboon uses by using the human sequence, and up to 75% reversible contraception was shown in 15 female baboons (O'Hern et al., 1995). A completely synthetic lactate dehydrogenase-C4 peptide vaccine consisting of just 39 amino acids coupled to a “promiscuous” T-cell epitope from tetanus toxin was capable of reducing fertility by 62% in female baboons (O'Hern et al., 1997). A promiscuous T-cell epitope is a short sequence capable of providing a T-helper response needed for antibody production in a wide range of species and multiple genetic backgrounds.

Therefore, full length as well as antigenic fragments of the lactate dehydrogenase-C4 (LDH-C4) antigen can be incorporated into the immunocontraceptive of the present invention. Table 1 shows a comparison of amino acids 1-15 of LDH-C4 from several species. From this table, it can be inferred that this particular sequence is divergent enough to provide some level of species specificity.

TABLE 1

Lactate Dehydrogenase-C4 Amino Acids
1-15 From Several Species
Amino acid differences between mouse
LDH-C and other species are underlined.

| | | |
|---|---|---|
| Mouse: | QLINLVPEDKLSR | (SEQ ID NO:18) |
| Rat: | QLINLAPDEKQSR | (SEQ ID NO:19) |
| Human: | QLIKLIEDDENSQ | (SEQ ID NO:20) |
| Hamster: | QLINLTQEDKTSR | (SEQ ID NO:21) |
| Pig: | QLINLIEDEVSQR | (SEQ ID NO:22) |
| Fox: | QLENLIEEDKISQ | (SEQ ID NO:23) |
| Pigeon: | QLIMTPITAQIKV | (SEQ ID NO:24) |
| Killifish (Retina): | HKLITPLACSSPE | (SEQ ID NO:25) |
| Frog: | NLITNVCQDKAAA | (SEQ ID NO:26) |

As used herein, “fragment,” as applied to a polypeptide, will ordinarily be at least 8 residues, more typically at least 40 residues in length, but less than the entire, intact sequence. Fragments of the lactate dehydrogenase-C4 protein can be generated by methods known to those skilled in the art, e.g., by enzymatic digestion of naturally occurring or recombinant lactate dehydrogenase-C4 protein, by recombinant DNA techniques using an expression vector that encodes a defined fragment of lactate dehydrogenase-C4, or by chemical synthesis.

Fragment of lactate dehydrogenase-C4 have been synthesized in combination with additional elements such as diptheria toxoid or a promiscuous T-cell epitope of tetanus toxin and shown to have comparable contraceptive effectiveness to lactate dehydrogenase-C4 in several species (O'Hern et al., 1995, 1997). Other carriers considered include the use of complete cholera toxin, its beta subunit, or a genetically or chemically detoxified form of the toxin. This agent has been shown to be the strongest mucosal adjuvant and can be more adequately used when oral and mucosal vaccines are considered. In a similar fashion, mucosal adjuvants from bacterial toxins or other sources could be used (Piazza, 2001).

One of ordinary skill in the art would recognize that the present invention is equally applicable to other animal or pest populations. Human activities, while driving some species to extinction or the brink of extinction, have provided a safe haven for many other species to proliferate to levels where they can pose great danger. Some of these species have become a menace to local flora, fauna, or, paradoxically, to humans themselves. There are multiple examples in many parts of the world where mammals have reached pest densities. For example, the white-tailed dear (Odocoileus virginianus) has been a menace in North America for almost a century. Many authors have proposed and tested the use of contraceptive vaccines for controlling vertebrate pests including deer. In fact, many vertebrate pests share many characteristics with the rodents and most models developed for their immunocontraceptive control can be equally applied. Thus, other animals to which the present invention is applicable include any mammalian species capable of eliciting an immune response against proteins of their own reproductive system. In particular, deer, elephants, water buffalo, feral horses, foxes, urban or wild dogs, urban or wild cats, rabbits, and other potentially overpopulated species causing economic damage to society could be targeted.

Crucial to the development of a successful contraceptive bait is the selection of a proper carrier for the immunological agent. The carrier should be stable, capable of being inexpensively and efficiently produced, environmentally safe and attractive as a food to the targeted animals. Based on these requirements, the present invention discloses a DNA vaccine that could be orally accessible using an attenuated bacterium. The contraceptive agents could be prepared into a bait formulation that could be strategically placed among the targeted animal populations. The animals would be sterilized upon consumption of the vaccine, thus promoting population control.

DNA Vaccine

In general, DNA vaccines consist of a plasmid with a strong mammalian promoter (usually a viral promoter), an antigenic gene, a polyadenylation sequence, and a termination signal. The plasmid is grown in bacteria, purified, and administered to a host by a variety of methods, usually by injection in saline or in colloidal gold using a “gene gun”. The cells take up the plasmid and the antigenic protein is endogenously produced. This leads to the production of antibodies and cell-mediated immunity specifically against the antigen, thus protecting the host against the antigen.

The first demonstration that DNA vaccines can be successful in providing protective immune responses was provided by immunizing mice with DNA encoding the conserved nucleoprotein from influenza A. Not only did vaccination with the plasmid induce the production of nucleoprotein specific antibodies and cytotoxic lymphocyte responses but also it provided protection when the animals were challenged with lethal doses of influenza. The extraordinary number of publications to date shows the versatility and robustness of this novel technology. In addition to viral diseases, other antigens from bacteria, parasites, tumors, allergy, and other antigens have been employed. However, no reports of an effective DNA contraceptive vaccine have yet been described.

It is now well established that epidermal, mucosal, intramuscular, and intravenous administration of plasmid DNA can induce both humoral and cell-mediated immune responses. Antigen-specific antibodies and cytotoxic T cell responses have been documented in rodents more than one year after immunization (Pardoll and Beckerleg, 1995). Depending on the route of delivery, many other tissues can take up plasmids, including the epidermis, dermis, liver and lymphocytes (Cheng, 1993).

Advantages of Genetic Vaccination

DNA vaccination possesses several advantages that make this strategy desirable. A valuable benefit is that genetic vaccination produces antigens that are identical to proteins produced endogenously by viruses and self proteins. This is because the antigens undergo the same treatment as native products, such as post-translational modification and cellular localization. This is important because recombinant proteins and other vaccination methods do not show, in some instances, comparable immunity.

A second advantage is that bacterial DNA is shown to provide an immunomodulatory effect by itself. Certain sequences of bacterial origin contain the unmethylated CpG motifs known to activate host defense mechanisms. DNA vaccination could eliminate the use of adjuvants, which often exhibit high toxicity, in a number of vaccination strategies. This is of particular importance when eliciting an immune response against factors that typically exhibit low immunogenicity, as is the case of self antigens in cancer, autoimmunity, and immunocontraception. Long-lasting persistence of plasmids in vaccinated individuals results in sustained synthesis of the antigens. This property will eliminate the need for boosts in some cases and will drive the induction of memory cells. Additionally, genetic immunization is able to overcome the unresponsiveness of immature lymphocytes that typically do not respond when other methods are used.

Other advantages exist that make DNA vaccines a more practical alternative for wide distribution than current vaccine technologies. DNA is more stable than both live attenuated vaccines and subunit vaccines in current use, eliminating the need for special treatments such as refrigeration. Moreover, the manufacture of plasmids is considerably cheaper than other vaccination methods. In addition, the amount of plasmid necessary to induce an immune response is generally small (on the order of micrograms).

When there is a need to develop vaccines against pathogens exhibiting natural genetic variation, the vectors can easily be modified, adapting the antigen by modifying DNA sequences. In addition, multiple genes expressing an array of epitopes could be used as a single vaccine component that can specifically target a desired response such as B or T cell stimulation. Examples of chimeric genes used to enhance immunity and provide protection have been reviewed in detail by Bona and Bot (2000).

Delivery of DNA Vaccines to Mucosal Sites

Genetic vaccination at mucosal surfaces, such as the respiratory system, can induce both mucosal and systemic immunity. In addition, mucosal immunization at one site has been shown to provide protection at distant mucosal sites. For example, lung vaccination has been shown to provide protection in vaginal areas. This property makes mucosal vaccination a versatile and convenient system that can be exploited to deliver DNA vaccines.

The delivery of genetic vaccines at mucosal sites has been pursued. These include noninvasive methods, direct injection, gene gun, and live attenuated bacteria (McCluskie and Davis, 1999). One promising approach is the use of attenuated bacteria as carriers of DNA vaccines.

The idea behind the delivery of DNA vaccines by attenuated bacteria lies in the fact that the microorganism can invade or be phagocytized by host cells. Once inside the cells the bacterium escapes the phagocytic vesicle using a series of mechanisms involving various virulence factors. After the organism breaks open into the cytoplasm, it dies due to previous genetic modifications that cause its attenuation (although, the infectivity mechanisms are kept intact). This causes release of the plasmids into the cytoplasm. The plasmid then reaches the nucleus by a poorly defined mechanism. The plasmid is episomally transcribed, the mRNA is transported to the cytoplasm, and the antigen gets expressed and modified.

The use of bacteria as a vehicle for plasmid vaccines should solve some of the problems of DNA vaccination and provide additional benefits. One of the advantages is that it can act as a powerful natural adjuvant by two distinct mechanisms. Since the bacteria target the inductive sites in the host, the antigen gets expressed at the most effective places. Secondly, bacterial cell wall and the unmethylated CpG bacterial sequences are capable of activating innate immune response.

Usually the plasmids used in DNA vaccination have high copy number and each bacterium can possess hundreds or even thousands of copies. This is particularly interesting since in the event that the bacterium is short lived and does not invade the cells, its lysis might provide the surrounding tissues with enough plasmid to elicit an immune response. The use of bacteria is convenient since their production, storage, and transportation is practical and cost effective. Lastly, bacteria can be treated with ordinary antibiotics in the case of an adverse reaction.

To date, the use of bacteria as carriers of DNA have been concentrated mainly on several species of Salmonella (typhimurium, typhi, flexneri) and to a lesser extent Lysteria monogenesis, Yersinia, Shigella, and BCG.

Salmonella typhimurium as a Carrier for DNA Vaccines

For the sole reason that mice are a natural host of S. typhimurium, more is known about the role this bacterium plays in delivering DNA vaccines than any other bacterial species. After the bacterium is orally delivered, it penetrates the intestinal wall through the M cells. This enables the bacteria to infect professional antigen presenting cells located immediately below the epithelium and leads to an inflammatory response. This is followed by Salmonella-specific antibody and cell mediated immune responses.

Metabolically attenuated Salmonella strains have been developed that retain their infectivity and immunogenicity but do not lead to infection. This makes the bacteria extremely useful not only for vaccination purposes but also as targeted transporters of heterologous antigens and plasmid DNA. The most common strains are deficient in the machinery that synthesizes aromatic amino acids and can only reproduce for several generations.

The first report of attenuated S. typhimurium used as an oral vehicle for a genetic vaccine was described by Darji et al (1997). Here, the bacterium was used to deliver eukaryotic expression vectors containing truncated constructs from ActA and listeriolysin, two virulence factors of Listeria monocytogenes. The group showed that even a single oral administration was capable of eliciting not only specific antibody and strong cytotoxic T cell responses but also provided protection against a lethal challenge. Subsequently, the versatility of this system was reported in a number of publications that showed successful uses with a number of viral, bacterial, fungi, and tumor antigens. A particularly interesting report was provided by Darji et al (2000) where a comparison of different S. typhimurium systems is provided. In this manuscript, it is discussed that a more invasive microorganism can be generated that can transmigrate M cells more actively thus providing a stronger immune response.

DNA Vaccines as Immunocontraceptives

The potential of DNA vaccine technology for immunocontraception is suggested by the demonstration that significant antibody levels can be obtained in vaginal secretions following a local inoculation with a model DNA-based antigen delivered by a gene gun. However, gene gun delivery is not a feasible approach for the control of mammals, in particular rodents. Therefore a more pragmatic solution is needed.

Perhaps the biggest advantage of DNA-based immunocontraceptive is that the antigen produced will have native conformation and post-translational modifications in contrast to antigen produced in bacteria. This can be critical with certain auto-epitopes, especially when the production of antibodies is required. Additionally, antigen that is produced endogenously eliminates the need for purification, stabilization, and other requirements that protein antigens require, thus significantly reducing costs, particularly when Salmonella is used to transport DNA.

Model contraceptive vaccines have been primarily focused on inducing serum antibody responses and little evidence exists on mucosal immunization. A DNA-Salmonella hybrid vaccine would not only be able to elicit a systemic humoral response but, based on previous evidence, it could also stimulate the mucosal branch providing adequate antibody titers in the reproductive tract. This is of particular importance since past reports have not been able to detect a direct correlation between humoral antibodies and infertility.

The first report of a DNA vaccine for contraceptive purposes was published by Rath et al. (2002). Here the authors used a construct containing the entire sequence of the egg antigen zona pellucida B (ZPB). Immunization of male BALB/cJ mice with ZPB DNA elicited significant antibodies that were able to bind to the native protein in a hemizona assay. (The hemizona assay is a test to determine the ability of the sperm to penetrate the egg). These preliminary results showed the feasibility of DNA vaccines to be used for immunocontraception.

Accordingly, the present invention is directed to a genetically modified bacterial host that expresses an immunocontraceptive comprising an egg- or sperm-specific polypeptide or antigenic fragment thereof. The sperm-specific polypeptide includes rat or murine lactate dehydrogenase-C. Representative polypeptides of murine lactate dehydrogenase-C include EQLIQNLVPEDK (amino acids 5-17; SEQ ID NO. 1), GLADELALVDADTDK (amino acids 44-58; SEQ ID NO. 2), GEALDLDQHGSLFLSTPK (amino acids 61-77; SEQ ID NO. 3), LGVNPTSCHGWVLGEHGDSSVPIWSGVNVAGVTLK (amino acids 180-210; SEQ ID NO. 4), SLNPAIGTDK (amino acids 180-210; SEQ ID NO. 5), QVVEGGYEVLDK (amino acids 231-243; SEQ ID NO. 6), EEVFLSIPCVLGESGITDFVK (amino acids 283-306; SEQ ID NO. 7), VNMTAEEEGLLLK (amino acids 307-316; SEQ ID NO. 8) and RMVSGQTRLDLLQR (amino acids 101-115; SEQ ID NO. 9) (Hogrefe et al., 1987).

Examples of egg specific polypeptides include egg zona pellucida glycoproteins 1, 2, and 3, e.g. PVTQSGPLRLELRLATDK (SEQ ID NO. 10), FGIHGPR (SEQ ID NO. 11) (Skinner et al., 1999; Sadler et al., 2000). Other applicable immunocontraceptive polypeptides include LNSSSSQFQIHGPR (SEQ ID NO. 12), CPKPDHTVTPDFYLAPPTTPEPFTPHAALHPIPDHTLAGSGHTGLTTLYPEQSFIHPTPAPPSLGPGPA (SEQ ID NO. 13), WFLQSDNEDARIHSLYGMISC (SEQ ID NO. 14), ALNNRFQIKGVELKS (SEQ ID NO. 15), NCAYKTTQANK (SEQ ID NO. 16), CQADSGLQNRLALFTFPNISETNVTYLFGHEENSTEHAMKGVC (SEQ ID NO. 17) (Hardy et al., 2002).

These contraceptive agents are useful in controlling the size of an animal population by inducing sterility in animals that have ingested the genetically-engineered bacteria. In general, the bacterial hosts include Salmonella typhimorium strains SL3261 or SL7202, other Salmonella species, Yersinia enterocolitica, Shigella flexner, Listeria monocytogenes, and recombinant Escherichia coli.

The present invention is also directed to methods of using these contraceptive agents to decrease the fertility of an animal. The bacteria could be freeze-dried or be prepared through other methods as a bait formulation. Alternatively, the bacteria can be washed and administered to the animals in the wild with the purpose of spreading the bacteria from animal to animal. Susceptible animals include mice, rats, deer, elephants, water buffalo, feral horses, foxes, urban or wild dogs, urban or wild cats, rabbits, and other potentially overpopulated species causing economic damage to society.

The following examples are given for the purpose of illustrating various embodiments of the invention and are not meant to limit the present invention in any fashion. The present examples, along with the methods, procedures, treatments, molecules, and specific compounds described herein are presently representative of preferred embodiments. One skilled in the art will appreciate readily that the present invention is well adapted to carry out the objects and obtain the ends and advantages mentioned, as well as those objects, ends and advantages inherent herein. Changes therein and other uses which are encompassed within the spirit of the invention as defined by the scope of the claims will occur to those skilled in the art.

### EXAMPLE 1

Construction of DNA Vaccine Vector

The creation of a DNA contraceptive required the construction of appropriate plasmid vaccine constructs. In this case, two vectors were considered: one encoding the entire cDNA sequence of mouse lactate dehydrogenase-C and one encoding a derived antigenic peptide of lactate dehydrogenase-C. The vector of choice was pcDNA3.1 since it features a strong mammalian promoter from the cytomegalovirus and other features that increase gene expression in mammalian cells.

pcDNA3.1-LDH-C

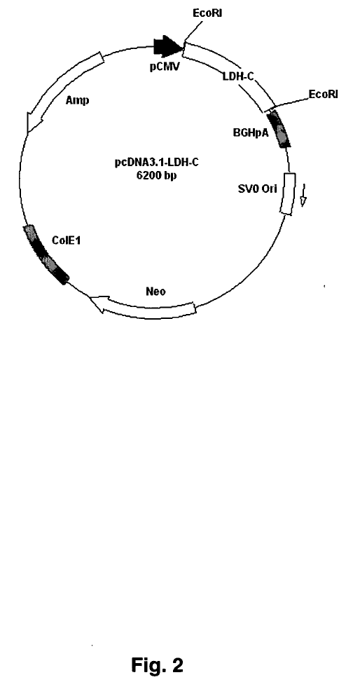

The pcDNA3.1-LDH-C, designed to express the compete lactate dehydrogenase-C sequence, was provided by Dr. Erwin Goldberg from Northwestern University and no further modification was performed with this construct. A map of the vector is shown in FIG. 2. This plasmid contains the cDNA of mouse lactate dehydrogenase-C between two EcoRI sites. The plasmid was transformed into XL-1 blue electrocompetent cells (Stratagene). The plasmid was isolated using the Quiaprep Spin Plasmid kit (Quiagen) following the manufacturer's instructions.

pcDNA3.1-SPV

A second construct, pcDNA3.1-SPV, was designed to express an immunodominant LDH-C epitope. SPV (Short Peptide Vaccine) was generated by a multi-step PCR using long oligonucleotides and then the gene was cloned into the appropriate vector. In the first step, a plasmid with the intact SPV region was used as a template for PCR so that the product comprises solely of the SPV sequence. The sequence of this minigene includes amino acids 5-15 of lactate dehydrogenase-C coupled via a short spacer to a promiscuous T-cell epitope from tetanus toxin. It does not contain a starting ATG codon. By using PCR, the first 5 amino acids of lactate dehydrogenase-C (amino acid 1 being an ATG) could be included in the sequence of SPV. This new minigene, containing amino acids 1-15 of lactate dehydrogenase-C, makes a more “natural” peptide vaccine in addition to including a start codon. The PCR product was then cloned into the pCR2.1 (Invitrogen, Ca) TA vector. This vector is conveniently designed for cloning PCR products easily without purification. Small fragments such as the SPV can be conveniently cloned and the complete procedure takes 2-3 days. The decision to use pCR2.1 in this instance was made because many researchers have encountered numerous difficulties trying to clone PCR products, in particular when these are as small as SPV.

A clone from the pCR2.1 ligation reaction was sequenced with the purpose of confirming its integrity and then it was submitted to restriction digestion with EcoRI. The product from the digestion was eventually inserted into the pcDNA3.1 to make a second DNA vaccine candidate. Plasmid DNA was prepared from several colonies from the overnight ligation reaction and these were further submitted to EcoRI digestion for screening purposes. The overall product, pcDNA3.1-SPV, was used subsequently in vaccine trials as a potential species-specific immunocontraceptive.

### EXAMPLE 2

Expression of DNA Vaccine Vector In Vitro

Before going into animal vaccine trials, attempts were made to express the antigen in vitro by transfecting the vectors into mammalian cells and testing for expression. The first step in the experiment was to develop a standardized protocol for plasmid transfection. This was accomplished by using the appropriate cell lines and the appropriate vectors. The cell line chosen was the COS-7 cells. This mammalian cell line has been designed to maximize protein expression in transfection experiments and has been widely described in numerous successful transformations.

For the control, plasmid pcDNA3.1-GFP (green fluorescent protein) was chosen since not only is the vector identical to the vaccine candidates, but direct visualization of protein expression can be easily performed and quantified under an epifluorescence microscope.

The transfection method that was used involved the use of lipid-DNA complexs; in particular, the Lipofectamine reagent from Gibco BRL. Although other methods were perfectly acceptable, the Lipofectamine protocols have been well established especially for COS-7 cells. In addition, many samples can be transfected at the same time since the preparation of the lipid-DNA complexes is extremely easy.

For this experiment, large quantities of highly purified plasmid DNA were needed. Cesium chloride gradients were used for this purpose and virtually limitless (˜10 milligrams) amounts of nucleic acids were obtained without detectable impurities.

For transfecting COS-7 cells with lipid-DNA complexes, several trials were performed with various amount of plasmid and Lipofectamine reagent to obtain optimal results. Every other variable such as volume, number of cells, confluency, and FCS concentration were kept constant in every attempt. The highest transfection efficiency for the pcDNA3-GFP was obtained when 6 μl of the Lipofectamine reagent and 10 μg of plasmid (at a concentration of 10 mg/ml) were used. In this case, over 90% transfection efficiency was obtained. This was calculated, using an epifluorescent microscope, by counting the number of cells that clearly fluoresced versus those that did not when exposed to UV light.

### EXAMPLE 3

Expression of DNA Vaccine Vector in Salmonella

Both constructs described above (pcDNA3.1-LDH-C and pcDNA3.1-SPV) were considered as the vaccine candidates to be tested. The pcDNA3-GFP was used as a negative control. The plasmids were transformed into Salmonella typhimorium strain SL3261. The Salmonella were subsequently grown without aeration and with high salt concentrations (regular LB medium plus 1.5%). Under these conditions, the Salmonella up-regulate the expression of a series of genes in the pathogenicity islet portion of the chromosome that help the bacteria increase its infectivity. This has the effect of making better vaccine candidates, as the attenuated bacteria become more intrusive without turning more pathogenic. Salmonella that has been grown under this environment will emit a strong putrid odor characteristic of infective bacteria.

Under these conditions, the Salmonella take several days to achieve acceptable numbers of bacteria in the cultures. This decreases the amount of plasmid since the ampicillin concentration needed to keep the bacteria under selection pressure gradually decreases. The option of supplementing the culture with additional ampicillin is not adequate nor it is to exchange the medium several times. Therefore, an alternative procedure was devised. The Salmonella were grown overnight with aeration until the culture was very dense. The next day, the cells were harvested and resuspended in fresh LB supplemented with sodium chloride and the antibiotic. No aeration was provided for 4 to 5 hours. The bacteria at that point started emitting the characteristic fetid odor. The cells were harvested, washed in Hanks' balanced solution, and resuspended in a minimum volume of medium. A pinch of sugar (sucrose) was added to make the formulation more flavorsome for the mice. Bacteria were counted by consecutive dilution and plating in ampicillin containing plates such that only the cells containing the plasmid were accounted for.

### EXAMPLE 4

Vaccine Preparation

A positive colony from the transformed. Salmonella was grown into a 50 ml LB medium culture supplemented with 50 mg/L of ampicillin overnight at 37° C., shaken at 200 rpm. Next day a 1 L culture was started under the same conditions and was grown overnight. The cultures were centrifuged at 6000 rpm in 500 ml sterile tubes for 10 minutes, the supernatant was discarded and the pellet was resuspended in 1 L of LB medium supplemented with 1.5% NaCl and 50 mg/L of ampicillin. The flasks were sealed with a plastic plug so that the culture would be grown under anaerobic conditions. A high salt concentration and low oxygen concentration is believed to up-regulate the expression of genes that will render a more pathogenic bacterium. The transformed Salmonella were incubated for 5-6 hours at 37° C. with minimal agitation (50 rpm). The cells were harvested by centrifugation as above and washed twice with Hank's balanced solution, pH 7.0, with some sucrose added to sweeten the solution and make it more palatable. The pellet from the last wash was resuspended in a minimum amount of buffer and the concentration of bacteria was adjusted to give approximately 109 colony forming units in 300 ul. A Qiaquick plasmid prep was performed on the Salmonella at this stage to check the integrity of the plasmid.

### EXAMPLE 5

Animals and Vaccine Administration

Six-week-old female Balb/c mice were purchased from Charles River Laboratories, caged in groups of 4 or 5, and housed at the University of Texas Animal Resource Center facilities. Water and food were removed from the cages 5 hours prior to vaccine administration. Normal feeding followed but each water bottle contained 1 g/L of ampicillin. This was done since, from previous experience, such Salmonella levels (even attenuated strains) made the animals very sick and this sickness was relieved by ampicillin. The vaccine was prepared the day of the administration and was given to the animals by gavage using a blunt-ended stainless steel needle.

### EXAMPLE 6

Fertility Assessment

After an adequate antibody response was detected, the animals were mated by introducing either two or three females into cages containing a single male. The males were previously tested for fertility and all the animals proved fertile. Once a female was impregnated, she was returned to her original cage. A female was considered impregnated only if she exhibited a clear post-coital vaginal plug. Under these mating conditions, all females were impregnated in 8 days, which includes at least 2 estrous cycles.

Fertility was tested by counting fully formed fetuses before the animals produced offspring. This is because it is not uncommon (it is actually expected) that adult mothers will devour some of the newborns. Mice have a gestation period of 19-21 days. The animals were sacrificed before the end of such a period (at two and a half weeks) and the litter size was determined by counting the number of fully sized animals.

### EXAMPLE 7

Blood Collection and Vaginal Washes

To measure antibody responses in serum and vaginal secretions, the animals were bled and vaginally washed on selected days. To prepare the serum, the mice were first restrained, their tail cut at the tip, and a few drops (7-10) of blood were collected in a tube for each animal. The blood was incubated for 20 minutes at 37° C. and then at 4° C. overnight to complete the clotting process. The blood was spun down in a microcentrifuge at 10000 rpm for 10 minutes. The serum was carefully removed, transferred into a fresh tube, and was either immediately used or kept frozen at −80° C.

Vaginal washes were performed by inserting a blunt pipette tip containing 50 μl of phosphate buffer saltine into the vagina and moving the liquid up and down 10 times. The wash was transferred into a microtube and was kept immediately at 4° C. and stored under the same conditions overnight where the large particles were allowed to sediment to the bottom of the tube.

### EXAMPLE 8

Indirect ELISA Assays

Production of specific antibody against lactate dehydrogenase-C in serum and vaginal washes was assessed by indirect ELISA. Briefly, 50 μl of 0.01 mg/ml recombinant lactate dehydrogenase-C in capture buffer (100 mM sodium bicarbonate pH 9.2) was coated onto 96-wells microtitration plate (Flow Laboratories, McLean Va.) and were further incubated overnight at 4° C. The plates were washed 4 times with PBST (phosphate buffer saltine pH 7.4, 0.5% Tween 20 (Sigma)), incubated at room temperature in blocking buffer (PBST with 1% protease-free BSA (Roche Biochemicals)) for 30 minutes and washed again once. Serum or vaginal washes were added to the wells up to 50 μl in blocking buffer and the plates were incubated for 1 hour at room temperature.

When IgA was tested both serum and vaginal washes were diluted 1:10. When IgG was tested serum was diluted 1:500 and vaginal washes were again diluted 1:10. The plates were washed 3 times as described above and then a secondary affinity purified goat anti-either mouse IgG or IgA coupled to a horseradish peroxidase was added. Both antibodies were obtained from Kirkegaard & Perry Laboratories (Gaithersburg, Md.). For IgG assessment, the antibodies were used at 1:10,000 dilution in blocking buffer. For IgA, a 1:1000 dilution was used. After one hour incubation at room temperature the plates were washed 4 times and 100 μl of hydrogen peroxide-2,2′-Azino-bis-(3-ethylbenzthiazoline-6-sulfonic acid (ABTS) from Moss Inc. (Pasadena, Md.) was added and incubated for 30 minutes in the dark. The reaction was quenched with 100 μl of 0.5 M oxalic acid. The plates were immediately read at 414 nm in an EL340 Automated Microplate Reader (Bio-Tek Instruments Inc., Winooski VM).

106

For IgA, the values for each individual mouse were tested in blood and vaginal washes. The mean along with the standard deviation was calculated and plotted using Sigmaplot™ 2001 v.7.0 software (SPSS Science, Chicago Ill.). In contrast, for IgG, the serum from each animal was pooled in equal amounts and was used for quantification. The IgG data was processed in Sigmaplot™ 2001 v.7.0 as above. The reason for testing pooled serum instead of each mouse individually was because the amount of serum was very limited, since tail bleeding produces only, at most, a couple of uls of serum.

### EXAMPLE 9

Measurement of T Cell Proliferative Responses

T helper cell (Th cell) proliferation assays were performed in order to determine the extent of activation that LDH-C and Salmonella confers and the efficacy of the vaccine to stimulate T cells. The assays were performed by a modification of the protocols by the Corradin et al. (1977) and Rosenwasser and Rosenthal (1998) references.

Mice were sacrificed by carbon dioxide asphyxiation and were immediately moved to sterile conditions. Spleens were removed and placed in 10 ml of RPMI 1640 (Invitrogen) supplemented with 2 mM L-glutamine, 1× antibiotic-antimycotic solution (Invitrogen), 50 μM β-mercaptoethanol, and 10% fetal calf serum (Invitrogen). The spleens were cut in small pieces and were then moved to a sterile 10 ml syringe. The syringe was connected to a 25 mm Easy Pressure Syringe Filter Holder (Gelman Laboratory, Ann Arbor, Mich.) and the cells were collected in a sterile 15 ml tube. From this point on, the cells were kept on ice.

Splenocytes were centrifuged at 1250 rpm for 10 minutes and the supernatant was removed. To remove erythrocytes that can further interfere with the assay, the pellet was resuspended in 5 ml sterile lysing buffer (150 mM NH₄Cl, 1.0 mM KHCO₃, 0.1 mM EDTA, pH 7.4) and incubated at room temperature for 5 minutes with occasional shaking. RPMI medium was added to fill the tube to 15 ml and the samples were centrifuged at 1250 rpm for 10 minutes. The supernatant was discarded and the cells were washed with media once more. The pellet was resuspended in 5 ml and splenocyte recovery was determined by counting the cells using a hemocytometer. The cell concentration was adjusted to 1×10⁶ cells/ml.

This Th cell assay needs cells that process the antigen but do not proliferate themselves causing unspecific proliferation. These antigen-presenting cells were prepared as splenocytes from untreated mice using the above protocol and then were treated with mitomycin-c that prevents cellular proliferation without killing the cells by intercalating in the chromosomal DNA. Once the splenocytes were obtained, they were centrifuged at 1250 rpm for 10 minutes and the pellet was resuspended in 2 ml of PBS. A fresh solution of mitomycin-c (Sigma) was prepared in PBS at 0.5 mg/ml and was kept in the dark at all times (the substance is extremely light sensitive). One hundred μl of the mitomycin-c solution was added to each ml of splenocytes and the cells were incubated at 37° C. for 20 minutes. The tubes were then filled to 15 ml with complete RPMI medium and centrifuged at 1250 rpm for 10 minutes and the supernatant was discarded. The wash was repeated two additional times. The pellet was then adjusted to 1×10⁶ cells/ml in complete RPMI.

Purified splenocytes (1×10⁵ cells) from vaccinated animals were plated in flat-bottom microwell sterile plates with or without 1×10⁵ mitomycin-c-treated antigen presenting cells. Recombinant lactate dehydrogenase-C (100, 10, or 1 μg/ml), heat-killed Salmonella (50, 10, or 1 μl) or no antigen as negative controls were also added to the wells. The recombinant lactate dehydrogenase-C was brought to 1 mg/ml with PBS, and was filtered-sterilized using a Millex-GV syringe driven filter unit with a 0.22 μm PDVF membrane (Millipore Co., Cork Ireland). Salmonella was grown in a 5 ml LB culture at 37° C. with constant agitation overnight started from a glycerol stock. The bacterium was heat killed by placing the culture in 65° C. water bath for 2 hours. The culture was cooled down to 4° C. and was diluted 1:10 in iced-cold sterile PBS before it was used in the assay. Each experimental group was set up in triplicates. The plates were covered and incubated at 37° C., 5% CO₂, and 90% humidity for 3 days.

Proliferation was quantified by a colorimetric cell proliferation ELISA, the BrdU colorimetric kit (Roche). In brief, the lymphocytes were incubated with the nucleotide analog 5-bromo-2′-deoxyuridine BrdU. The cells were harvest and fixed to the plates 20 hours latter. The unfixed material was washed and the microwells were probed with a monoclonal antibody against BrdU coupled to horseradish peroxidase. The solution was washed and then incubated with substrate solution for 20 minutes. The color development was stopped with 25 μl of 1M H₂SO₄. The plates were read at 450 nm in a Spectramax 190 microplate reader (Molecular Devices, Sunnyvale Calif.).

### EXAMPLE 10

First Vaccine Trial

For the first vaccine trials 25 female mice were used for experimentation: 10 for pcDNA3-LDH-C; 10 for pcDNA3-SPV; and 5 for pcDNA3-GFP. Four weekly doses and one boost at day 82 of 10⁸ colony-forming units were administered directly into the back of their throats forcing the formulation into their stomachs without causing extreme discomfort but ensuring systematic dispense. Two hours prior to each administration, water and food were removed from the cages. Fasting animals not only will have empty stomachs that can easily accommodate the bacteria but will be free from the normally hostile milieu needed for digestion.

The process of vaccine administration was proven to be a difficult one and lead to several unintended casualties. Some animals died due to Salmonella infection, since no laceration was detected. One animal that was previously vaccinated with pcDNA3-LDH-C Salmonella was unintentionally killed after the second administration (day 17). The corpse was immediately placed on ice and the DNA was isolated from the animal's stomach and spleen using a Dneasy™ Tissue Kit (Quiagen, Germany). The DNA was subsequently subjected to PCR using a T7 promoter primer (not contained in normal tissue) and a primer complementary to the 3′ end of the lactate dehydrogenase-C gene.

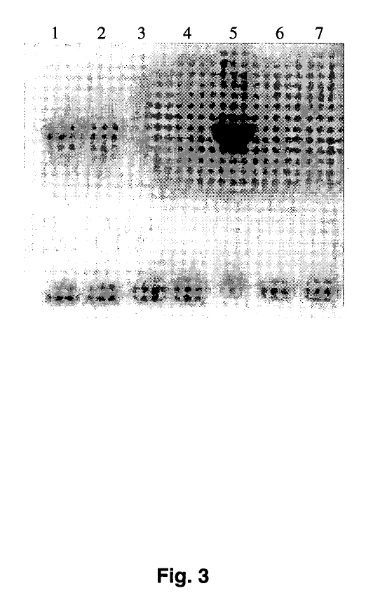

As can be seen in FIG. 3, the plasmid could be detected in the stomach but not in the spleen, which suggests that Salmonella never reached (at least at this stage) the spleen but was able to establish in the stomach. This could lead to the conclusion that, since the plasmid was present in the digestive system but not in the spleen, a more localized mucosal response would be elicited.

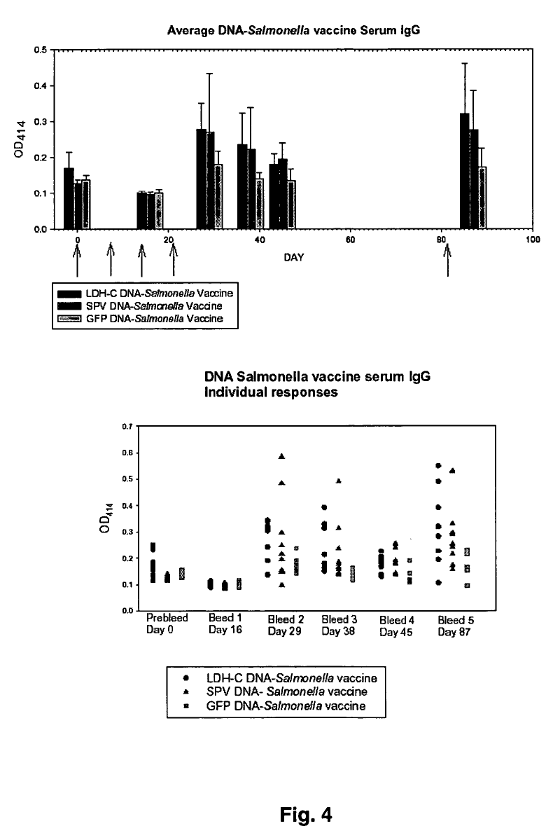

The immune system was constantly monitored by testing the antibody response in serum and vaginal washes. The antibody response was tested by indirect ELISA using recombinant lactate dehydrogenase-C to capture the specific antibodies. In particular, serum IgG and IgA and vaginal IgA were assessed. The animals were bled and washed on days 0, 16, 23, 29, 38, 45, and 87. ELISAs were performed for each bleed/wash and the results were plotted against OD₄₁₄. (FIGS. 4-6).

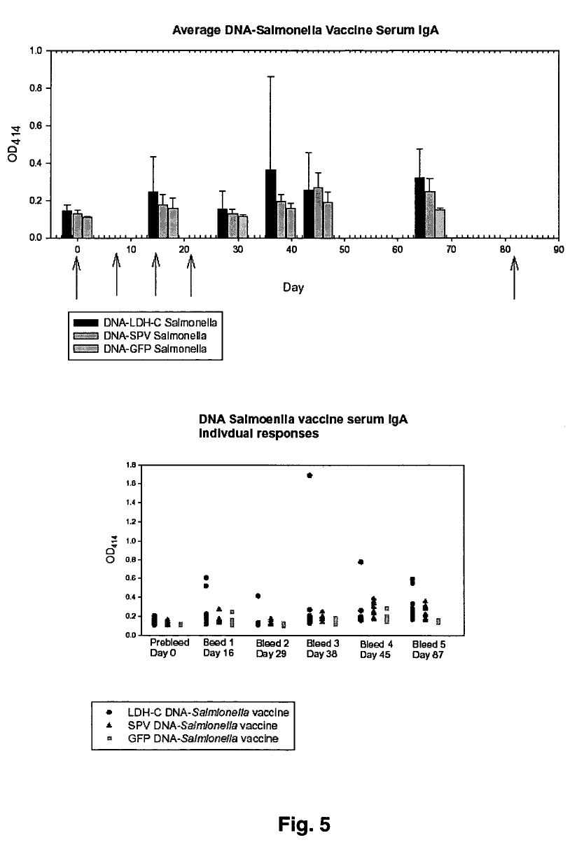

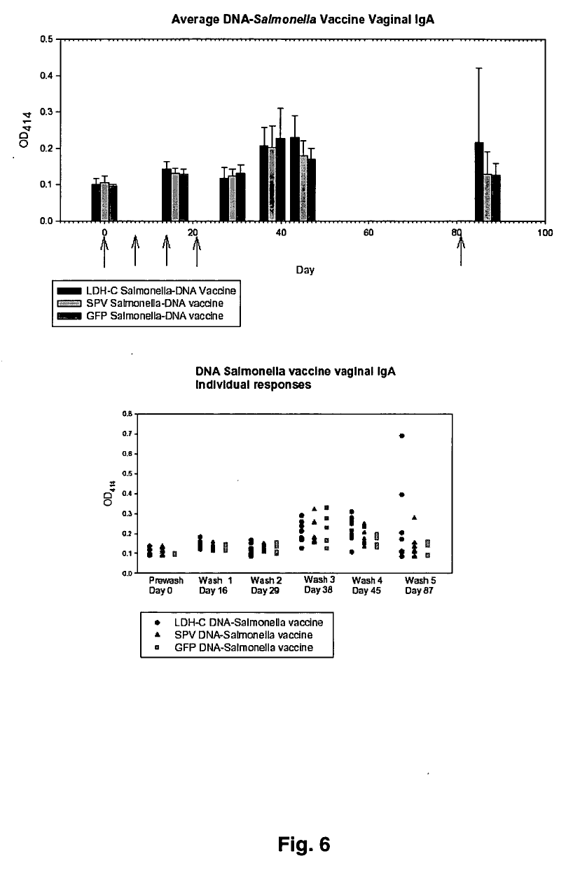

FIG. 4 shows that the pcDNA3-LDH-C and pcDNA3-SPV was able to elicit a modest but specific immune response compared to the control pcDNA3-GFP. A high background could be seen in some GFP bleeds and it is believed that this is due to bacterial lipopolysacharides which are highly immunogenic and can cause an allergic-like inflammation response and even death. Similar graphs illustrate serum and vaginal IgA are depicted in FIGS. 5 and 6, respectively.

These data show that the DNA vaccines were able to elicit antibodies in both vaginal fluids and serum. The average response for serum IgA seems adequate since this isotype is not typically present in large amounts in serum. That is why a dilution of 1:10 was needed to detect a response. For the vaginal washes, some IgA was also present according to the ELISAs. However, the response was not as consistent as the serum. This is, in part, because the process of collecting vaginal washes can be cumbersome and ample experience is needed to conduct systematic washes. Despite these difficulties, lactate dehydrogenase-C specific IgA was present in the vaginal area, which is highly desirable obviously for sperm neutralization. After the last boost at day 82 there was no increase in antibody titers for any isotype.

### EXAMPLE 11

Second Vaccine Trial

Results from the above vaccination trial show that mice are able to make an antibody-based immune response towards lactate dehydrogenase-C when Salmonella-DNA vaccine constructs were fed. The purpose of the present vaccine trial was primarily focused on testing the effects on fertility and to assess T cell proliferative responses.

An ideal contraceptive vaccine for overpopulation control would require minimum number of doses. This is true for any vaccination strategy. This trial was designed to account for this and ultimately minimize the number of doses.

For this experiment, 3 doses of 10⁹ colony-forming units (300 μl) were administered to Balb/c female mice on days 0, 7, and 47. The DNA vaccine constructs used were the same as in the previous vaccine trial. Eight mice were used for pcDNA3-LDH-C, six for pcDNA3-SPV, and six for the control pcDNA3-GFP. Keeping a small manageable population was needed to effectively perform adequate mating strategies.

The Salmonella carrying the plasmids was grown, as before, in LB supplemented with 1.5% sodium chloride without aeration for a few hours. To ensure that each preparation of Salmonella contained comparative amounts of plasmid, a sample of about 10⁸ bacteria from each sample was used for plasmid preparation using a Qiaquick Minipreps (Qiagen, Germany). The samples were run on agarose gel to confirms that approximately equivalent amounts of plasmid were given in each dose and the integrity of the plasmid was not compromised by the sample preparation used.

Immunoglobulin Responses

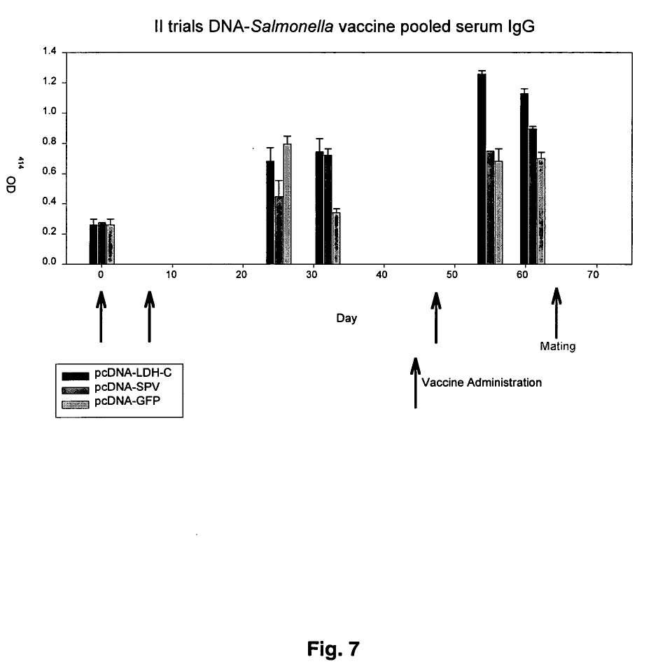

The animals were periodically monitored for the production of antibodies. As before, serum IgG and IgA and vaginal IgA were tested by indirect ELISA. After all the blood samples were collected, the serum from each group of individual mice was pooled and the samples subjected to ELISA. This was done with the purpose of testing the sample under identical conditions. A plot of these samples is shown in FIG. 7. In this example, the serum samples were incubated overnight at 4° C. instead of 1 hour at room temperature. This produced plots that were more suitable for analysis in spite of a background increase.

Results shown in FIG. 7 establish that the animals were responding to the vaccine by producing lactate dehydrogenase-C specific antibodies, at least when the pcDNA3-LDH-C was used. As for the pcDNA3-SPV construct, only a modest response was detectable in general compared to the control except for the second bleed.

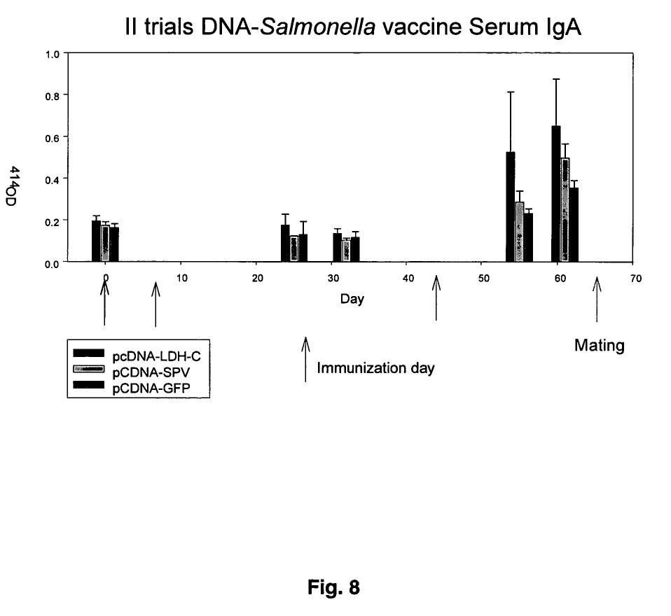

The serum IgA response was also tested using pooled serum and is shown in FIG. 8. From this graph it can be seen that the animals did not respond until a boost was administered. However, this response was satisfactory and more consistent than the IgG response.

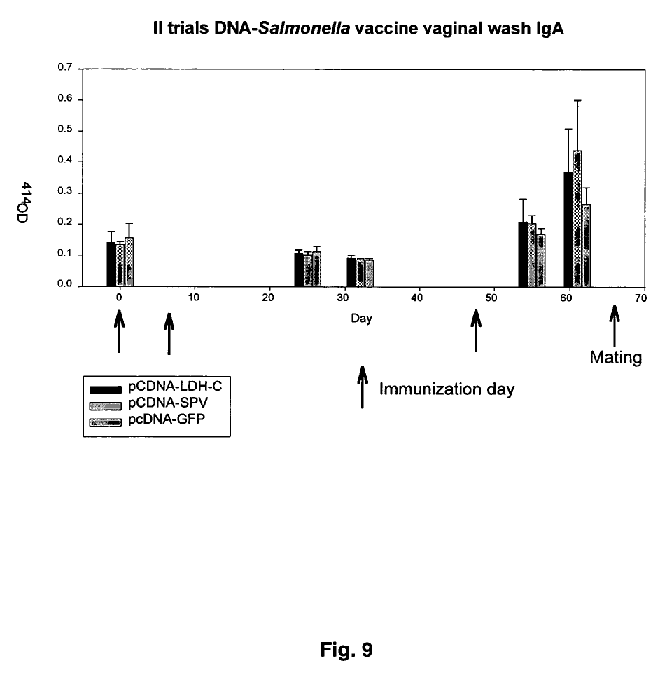

Lastly, the lactate dehydrogenase-C specific IgA in vaginal fluids was tested. For this, the washes were pooled which was a difficult and somewhat inconsistent task due to the viscosity of the fluids. A plot of these results is shown in FIG. 9. Consistent with the serum IgA response, the animals did not produce quantifiable amounts of the vaginal specific antibody until the third administration of the vaccine (which can be considered a boost).

Fertility Assessment

On day 65, after an adequate antibody response was detected, the animals were mated by introducing either two or three females into cages containing a single male. The males were previously tested for fertility and all the animals proved fertile. Once a female was impregnated, she was returned to her original cage. A female was considered impregnated only if she exhibited a clear post-coital vaginal plug. Under these mating conditions, all females were impregnated in 8 days, which includes at least 2 estrous cycles.

Table 2 shows that pcDNA3-LDH-C was able to reduce fertility in female mice from an average of 5.75 of the pcDNA3-GFP to 2. This corresponds to a reduction of about 65%. The recorded average first litter size for the Balb/c genotype is 5 pups per animal (Technical Bulletin #1, Charles Rivers Laboratory, Spring 1999). This is consistent with the average litter size found in mice treated with the peptide or control plasmids.

T Cell Proliferative Response

Antibody production is an exquisitely complex mechanism requiring the interaction of a variety of immune cells and signals originally stimulated by an antigen. In the case of immunocontraceptives, activated helper T-cells are responsible for stimulating antibody-secreting cells. The ability of a vaccine to stimulate Th-cells is essential for this particular vaccination strategy since there are many factors that can contribute to the activation of lymphocytes. In this case, it was unknown whether lactate dehydrogenase-C or the Salmonella would be the primary stimulators of T-cell response.

To determine the key players involved in stimulating a cellular immune response the ability of both the Salmonella and the lactate dehydrogenase-C was tested in vitro using splenocytes from the vaccinated or control animals. This was done using standard in vitro lymphocyte proliferation assays as described above.

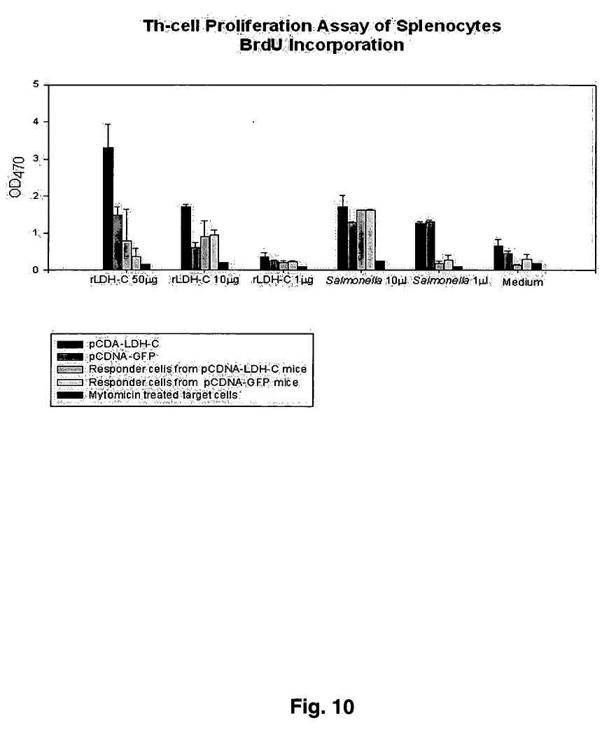

The day that the animals were sacrificed for fertility testing, their splenocytes containing Th-cells were isolated. Spleens from animals vaccinated with the pcDNA3-LDH-C or the control pcDNA3-GFP were used as responder cells. The responder cells were incubated with splenocytes from naive mice pre-treated with mitomycin C. Mitomycin C inhibits the ability of cells to proliferate but the cells retain the ability to process and present antigen. Both populations of cells were incubated in the presence or absence of antigen. The antigens tested were heat-killed Salmonella and recombinant lactate dehydrogenase-C. The splenocytes incubated in the presence of antigens for 3 days were then spiked with bromodeoxyuridine (BrdU) for 18 hours. If the splenocytes proliferate, the BrdU will get incorporated into their chromosomes. BrdU incorporation is proportional to the proliferation levels and it can be tested with a monoclonal antibody against the nucleotide in an ELISA setting. The results from the ELISA were plotted and shown in FIG. 10.

The results strongly suggest that lactate dehydrogenase-C was essential in stimulating a Th-response since it exhibited a dose dependent response. In fact it is known that the lactate dehydrogenase-C sequence contains a Th-cell epitope. Salmonella, on the other hand, did not seem to be as critical as suspected. This is because the signal did not increased in proportion to the amount of bacterium added to the assay. The noise (non-specific proliferation of responder cells without a target), however, increased as more antigen was added but the signal just reached a plateau. A large non-specific proliferation was expected since the Th-cells were not purified from splenocytes. The splenocytes include a number of antigen presenting cells (e.g. macrophages, monocytes, etc.) that can proliferate non-specifically in the presence of any known antigen. In summary, these results not only showed the importance of LDH-C in producing a helper response but also confirmed that Salmonella is an extremely efficient carrier of plasmid-based vaccines.

TABLE 2
Average fertility of DNA-Salmonella vaccinated Mice

| | | | Average |
|---|---|---|---|
| | Total | Total number | number of |
| Group | females tested | of fetuses | fetuses/female |
| pcDNA3-LDH-C | 5 | 10 | 2 |
| pcDNA3-GFP | 4 | 23 | 5.75 |
| Balb/c | | | 5-6 |
| (Charles Rivers Labs) | | | |

### EXAMPLE 12

Salmonella-DNA Vaccine as Immunocontraceptive

The data presented above indicate that administration of Salmonella-DNA vaccine in female mice resulted in ˜65% reduction in fertility. Such infertility has never before been achieved in mice with lactate dehydrogenase-C. This level of infertility (although only tested in a few mice) is by itself acceptable as a rodent control. Future experimentation will determine the efficacy of the vaccines in males and the effect the DNA-Salmonella hybrid will have when both sexes are treated with the vaccine. If 65% infertility could be achieved in females, it most likely would show much higher levels if both sexes are treated; perhaps complete sterility. Salmonella-DNA vaccine can induce such a high level of infertility because this vaccine was capable of inducing a high level of IgA (see FIGS. 8 and 9). These results emphasize the need for future contraceptive vaccines to target mucosal areas. Not only has IgA evolved to be an integral part of mucosal secretions and is more stable in the vaginal area, but its polymeric nature may be more effective in neutralizing sperms in vivo.

The fact that splenocytes were able to produce a robust T-helper response against lactate dehydrogenase-C but a only modest one against the Salmonella means that the antigen reached the spleen either intact or processed by professional antigen presenting cells. The main antibody isotype was IgA and the expression of lactate dehydrogenase-C was detected in intestine extracts. Based on these experimental evidences the following mechanism is suggested. The Salmonella reaches the epithelium and gets transported to the lymphoid tissue underneath, where it gains entry into the cells. The bacteria dies and the plasmid is shuttled to the nucleus where lactate dehydrogenase-C is eventually expressed. Lactate dehydrogenase-C is secreted in large enough quantities to stimulate the epithelial B-cells to produce IgA antibodies. At the same time, professional antigen presenting cells distribute the antigen to multiple lymph nodes where a T-cell response is stimulated. The validity of this mechanism could be corroborated with future experimentation. One such set of experiments is to test the invasiveness of the Salmonella. This can be accomplished by orally immunizing the animals and then extracting DNA from different tissues. The extracted DNA can be subjected to PCR using primers directed against either the plasmid or the bacterium's chromosome. The kinetics for the establishment of Salmonella and the production of antigen should also be tested. This can be accomplished by either PCR, RT-PCR, Western blots, or immunostaining for lactate dehydrogenase-C using different tissues at different times after administration.

The importance of recognizing the mechanism goes beyond a pure academic exercise. It is essential to recognize key players and the role they play in this vaccination strategy not only to make more effective vaccines but also to minimize (if possible) any components that may provide toxic effects to the target or non-target species. For example, it would be ideal if the Salmonella could be replaced by other organisms such as E. coli or even nonreplicating agents such as plasmid carriers. It should be stressed here that pathogens such as Salmonella are common dwellers in rodent colonies and rats and mice are carriers of several bacterial species. Furthermore, the introduced bacterium should be highly attenuated and cannot propagate beyond its host. In any case, systems that carry the plasmid without antibiotic resistance should be pursued if any form of DNA shall be used as a contraceptive vaccine. Some systems of antibiotic resistance-free plasmids are available.

The following references were cited herein:
Bona and Bot, Genetic Immunization. Kluwer Academic/Plenum Publisher. New York, N.Y. (2000).
Cheng et al., In vivo promoter activity and transgene expression in mammalian somatic tissues evaluated by using particle bombardment. Proc Natl Acad Sci USA. 90: 4455-9 (1993).
Darji et al., Oral somatic transgene vaccination using attenuated S. typhimurium Cell 91:765-75 (1997).
Darji et al., Oral delivery of DNA vaccines using attenuated Salmonella typhimurium as carrier. FEMS Immunol Med Microbiol. 27:341-9 (2000).
Goldberg, Infertility on female rabbits immunized with lactate dehydrogenase-X. Science 181:458 (1973).
Goldberg et al., Reduction of fertility in female baboons immunized with lactate dehydrogenase-C4. Fertil. Steril. 35:214 (1981).
Hardy et al., Reprod. Suppl. 60:19-30 (2002).
Hogrefe et al., Characterization of the antigenic sites on the refined 3-Å resolution structure of mouse testicular lactate dehydrogenase C4. J. Biol. Chem. 262:13155-62 (1987).
Kaumaya et al., Design and immunological properties of topographic immunogenic determinants of a protein antigen (LDH-C4) as vaccines. J. Biol. Chem. 267:6338-46 (1992).
Lerum and Goldberg, Immunological impairment of pregnancy in mice by lactate dehydrogenase-X. Biol. Reprod. 11:108 (1974).
McCluskie and Davis, Mucosal immunization with DNA vaccines. Micro Infec. 1:685-98 (1999).
O'Hern et al., Reversible contraception in female baboons immunized with a synthetic epitope of sperm-specific lactate dehydrogenase. Biol. Reprod. 52:331-39 (1995).
O'Hern et al., Vaccine 16:1761-6 (1997).
Pardoll and Beckerleg, Exposing the immunology of naked DNA vaccines. Immunity 3:165-9 (1995).
Pizza et al., Vaccine 21:2534-41 (2001).
Rath et al., Ecological basis for fertility control in the house mouse (Mus domesticus) using immunocontraceptive vaccines. Mol Reprod Dev. 62:525-33 (2002).
Sadler et al., The antigenic and immunogenic properties of synthetic peptide immunocontraceptive vaccine candidates based on gamete antigens. Vaccine 18: 416-25 (2000).
Skinner et al., Biol. Reprod. 61:1373-80 (1999).
Van Regenmortel et al., Immunol. Lett. 17:95-107 (1998).

Any patents or publications mentioned in this specification are indicative of the levels of those skilled in the art to which the invention pertains. Further, these patents and publications are incorporated by reference herein to the same extent as if each individual publication was specifically and individually indicated to be incorporated by reference.

1. A recombinant bacterial host useful as an immunocontraceptive vaccine, said bacterial host comprises a vector encoding an egg- or sperm-specific polypeptide.

2. The bacterial host of claim 1, wherein said polypeptide is linked to a sequence encoding an adjuvant.

3. The bacterial host of claim 2, wherein said adjuvant is selected from the group consisting of diptheria toxoid, the T-cell epitope of tetanus toxin, cholera toxin, beta subunit of cholera toxin, and detoxified form of cholera toxin.

4. The bacterial host of claim 1, wherein said sperm-specific polypeptide is lactate dehydrogenase-C or an antigenic fragment thereof.

5. The bacterial host of claim 4, wherein said lactate dehydrogenase-C is a rodent lactate dehydrogenase-C.

6. The bacterial host of claim 5, wherein said rodent lactate dehydrogenase-C is a rat lactate dehydrogenase-C or mouse lactate dehydrogenase-C.

7. The bacterial host of claim 4, wherein said antigenic fragment is selected from the group consisting of SEQ ID NOs. 1-9.

8. The bacterial host of claim 1, wherein said polypeptide is selected from the group consisting of SEQ ID NOs. 10-17.

9. The bacterial host of claim 1, wherein said bacterial host is selected from the group consisting of Salmonella typhimorium, Salmonella typhimorium strain SL3261, Salmonella typhimorium strain SL7202, Yersinia enterocolitica, Shigella flexner, Listeria monocytogenes, and Escherichia coli.

10. A method of decreasing the fertility of an animal, comprising the step of:
allowing said animal to ingest the bacterial host of claim 1.

11. The method of claim 10, wherein said animal is selected from the group consisting of mouse, rat, deer, elephants, water buffalo, feral horses, foxes, urban or wild dogs, urban or wild cats, rabbits, and other potentially overpopulated species causing economic damage to society.

12. The method of claim 10, wherein said bacterial host is freeze-dried or prepared as a bait formulation.

13. A recombinant bacterial host that expresses a sperm-specific polypeptide lactate dehydrogenase-C or an antigenic fragment thereof.

14. The bacterial host of claim 13, wherein said lactate dehydrogenase-C is a rodent lactate dehydrogenase-C.

15. The bacterial host of claim 14, wherein said rodent lactate dehydrogenase-C is a rat lactate dehydrogenase-C or mouse lactate dehydrogenase-C.

16. The bacterial host of claim 13, wherein said antigenic fragment is selected from the group consisting of SEQ ID NOs. 1-9.

17. The bacterial host of claim 13, wherein said polypeptide or antigenic fragment is linked to a sequence encoding an adjuvant.

18. The bacterial host of claim 17, wherein said adjuvant is selected from the group consisting of diptheria toxoid, the T-cell epitope of tetanus toxin, cholera toxin, beta subunit of cholera toxin, and detoxified form of cholera toxin.

19. The bacterial host of claim 13, wherein said bacterial host is selected from the group consisting of Salmonella typhimorium, Salmonella typhimorium strain SL3261, Salmonella typhimorium strain SL7202, Yersinia enterocolitica, Shigella flexner, Listeria monocytogenes, and Escherichia coli.

20. A method of decreasing the fertility of an animal, comprising the step of:
allowing said animal to ingest the bacterial host of claim 13.

21. The method of claim 20, wherein said animal is selected from the group consisting of mouse, rat, deer, elephants, water buffalo, feral horses, foxes, urban or wild dogs, urban or wild cats, rabbits, and other potentially overpopulated species causing economic damage to society.

22. The method of claim 20, wherein said bacterial host is freeze-dried or prepared as a bait formulation.
